(19) United States
(12) Patent Application Publication
(10) Pub. No.: US 2002/0177769 A1
(43) Pub. Date: Nov. 28, 2002

(54) Method and apparatus for locating cells in the body by measuring magnetic moments

(75) Inventors: Raymond L. Orbach, Washington, DC (US); John S. Kovach, Setauket, NY (US); Gregory G. Kenning, Riverside, CA (US)

Correspondence Address: John P. O'Banion, O'BANION & RITCHEY LLP, Suite 1550, 400 Capitol Mall, Sacramento, CA, 95814, US

(73) Assignee: THE REGENTS OF THE UNIVERSITY OF CALIFORNIA

(21) Appl. No.: 10/131,792

(22) Filed: Apr. 23, 2002

Related U.S. Application Data
(60) Provisional application No. 60/285,916, filed on Apr. 23, 2001.

(51) Int. Cl. A61B 5/05

(52) U.S. Cl. 600/409; 600/431; 128/899

(57) ABSTRACT

A magnetic body scanning method and apparatus for scanning the entire body for a magnetic signature of a cluster of ferromagnetic nanoparticles in relation to the diamagnetic signature of the body.

### CROSS-REFERENCE TO RELATED APPLICATIONS

This application claims priority from U.S. provisional application serial No. 60/285,916 filed on Apr. 23, 2001, incorporated herein by reference.

### STATEMENT REGARDING FEDERALLY SPONSORED RESEARCH OR DEVELOPMENT

Not Applicable

### REFERENCE TO A COMPUTER PROGRAM APPENDIX

Not Applicable

### BACKGROUND OF THE INVENTION

1. Field of the Invention

This invention pertains generally to imaging in the human body, and more particularly to a magnetic body scanning method and apparatus for scanning all or a portion of a human body for a magnetic signature of a cluster of ferromagnetic nanoparticles in relation to the diamagnetic signature of the body.

2. Description of the Background Art

Cancer mortality rates are directly correlated to the stage at which the cancer is first discovered. Therefore, much effort has gone into non-invasive physical analysis of the properties of matter in the body in order to image cancers in their early stages. For example, X-ray imaging (measuring electron density), ultrasound imaging (measuring the reflection coefficient of specific frequencies of sound), and Magnetic Resonance Imaging (measuring the decay rates of nuclei specific magnetic energy levels)have all found important places in the detection of cancer.

Ultra-sensitive Superconducting Quantum Interference Device (SQUID) amplifiers have been used in the past for imaging the magnetic fields produced by electrical activities in the brain and heart. This sensitivity, coupled with the new development of nanometer sized antibody-coupled superparamagnetic particles, now offers the possibility of imaging tumors by imaging the local magnetic field produced by a magnetization enhanced tumor (Enhanced Magnetization Imaging (EMI)).

Over the last two decades, dextran coated magnetic nanoparticles have found a variety of applications in the biological and medical sciences. Molday and Mackenzie described the use of dextran-coated particles with a 15 nm iron oxide core coupled to antibodies and other ligands for cell separation in the laboratory. See, Molday, R. S. and Mackenzie D., “Immunospecific Ferromagnetic Iron-Dextran Reagents For The Labeling and Magnetic Separation of Cells”, Journal of Immunological Methods, 52, 353-367 (1982), incorporated herein by reference. More recently, a variety of smaller particles, MIONS (Monocrystalline Iron Oxide Nanocolloid), PION (Polycrystalline Iron Oxide Nanocolloid), LCDIO (Long Circulating Dextran-coated Iron Oxide), and USPIO (Ultra Small Superparamagnetic Iron Oxide), have found application as contrast agents in MRI studies. See, Shen T., Weissleder R., Papisov M., Bogdanov A., Brady T. J., “Monocrystalline Iron Oxide Nanocompounds (MIONS) Physicochemical Properties”, Magn. Reson. Med 31, 599-604 (1994); Mandeville J. B., J. Moore, D. A. Chesler, L. Garrido, R. Weissleder and R. M. Weisskoff, “Dynamic Liver Imaging with Iron Oxide Agents: Effects of Size and Biodistribution on Contrast”, Magnetic Resonance Imaging:885-890 (1997); Moore A., Marecos E., Bogdanov A. and R. Weissleder, “Timoral Distribution of Long-Circulating Dextran-coated Iron-Oxide Nanoparticles in a Rodent Model”, Radiology 2000; 214:568-574; and Enochs, W. S., Harsh, G., Hochberg, R. Weissleder, R., “Improved Delineation of Human Brain Tumors on MR Images using a long circulating, superparamagnetic Iron Oxide agent”, Journal of Magnetic Resonance Imaging, 9 (2):228 (1999); respectively, each of which is incorporated herein by reference. These particles have been introduced in vivo and are found to be non-toxic. Maximum localization on a specifically targeted region (tumors, liver, lungs, lymph nodes) occur approximately 24 hours after injection and typically the majority of nanoparticles have passed out of the body within a week. Tumor cell uptake of LCDIO in a rodent model was found to be between 11.9 ng and 118 ng of iron per million cells. See, Moore A., Marecos E., Bogdanov A. and R. Weissleder, “Timoral Distribution of Long-Circulating Dextran-coated Iron-Oxide Nanoparticles in a Rodent Model”, Radiology 2000; 214:568-574. This is not an insignificant amount and opens the possibility of detection by ultra-sensitive SQUIDS.

The most commonly used magnetic nanoparticles for biological or medical applications are the beta phase of β-Fe₂O₃ (Magnetite) and the gamma phase of γ-Fe₂O₃ (Maghemite). Both Magnetite and Maghemite are different structural phases of ferrous oxide. Production of magnetic nanoparticles is relatively straightforward and several different procedures for making them are in the literature. See, Molday, R. S. and Mackenzie D., “Immunospecific Ferromagnetic Iron-Dextran Reagents For The Labeling and Magnetic Separation of Cells”, Journal of Immunological Methods, 52, 353-367 (1982). In general the dextran coated particles precipitate out of solution and the smaller particles are separated by liquid chromatography. Both Magnetite and Maghemite have a spinel structure (see, Shull C. E., Wallen, E. O. and Kochler, W. C., “Neutron Scattering and Polarization by Ferromagnetic Materials”, Phys. Rev. 84, 912-921 (1951), incorporated herein by reference) and internally the iron atoms in this structure interact magnetically with each other through an intermediary oxygen atom. This type of interaction is called a superexchange interaction and leads to ferrimagnetic behavior in the bulk material. Ferrimagnetic materials are characterized by a spin imbalance between the two antiferromagnetically interacting sublattices leading to a net local spontaneous magnetic moment in an applied magnetic field, which increases with applied field up to some characteristic saturation magnetization. See, “Magnetite Biomineralization and Magnetoreception in Organisms”, Ed. Kirschvink J. L., Jones, D. S. and MacFadden, B. J., Plenum Press (1985), incorporated herein by reference.

When considering Magnetite (Fe₂O₃) or Maghemite (Fe₃O₄) particles for magnetic labeling, particle size is a crucial issue. In general, particle size determines both the magnetic properties of the label and ability of the particle to move through the body. There are effectively three size dependent classifications for Magnetite (and similarly Maghemite). These include Bulk properties or Multi-Domain (MD) particles, Single-Domain (SD) and Pseudo Single Domain particles, and SuperParamagnetic (SP) particles.

In zero magnetic field a bulk sample or large particle (>10 μm) will break up into domains of magnetization, each with a net spontaneous magnetization. In zero magnetic field, in order to satisfy energy requirements, the domains will align to give a zero net sample moment. In a non-zero magnetic field the sample can have a net moment and will achieve this through domain wall motion and domain growth. The one feature of this type of behavior that may be of use in this study is that this type of behavior leads to a time-dependent remanent magnetization. Since the magnetic features in the body rapidly align their magnetization to the induced magnetic fields a slowly varying remanent magnetization would provide another dimension for identification of the particles. While the crossover to multidomain structures occurs for sizes of magnetite that are almost certainly too large for useful introduction into the body (with the possible exception of the lungs), the remanent properties of any of the possible magnetic labels must be considered.

Single Domain or Pseudo Domain particles range in size between 25 nm and 10 μm. These particles have a single domain and hence a net spontaneous magnetization. The magnetization is aligned along an easy magnetization axis of the crystal generating a strong anisotropy in the sample. In an external magnetic field the particles will try to align in the field however alignment would require alignment of the anisotropy axis and hence a rotation of the particles as a whole. This process is very expensive in energy and leads to Single Domain Particles having a large coercive field.

The first measurement of the magnetic fields generated in the body was performed in 1963 by Baule and McFee, who measured the magnetic field of the heart (magnetocardiogram) using an induction coil magnetometer. See, Baule G. M. and McFee R., “Detection of the Magnetic Field of the Heart”, Am. Heart J. 55:(7), 95-96 (1963), incorporated herein by reference. Since that time, new technologies have evolved increasing the sensitivity by over four to five orders of magnitude over induction coil techniques. This sensitivity opens the possibility of measuring and imaging, at a distance (non-invasively), the weak magnetic fields generated by the electrical activity in the brain and heart. In general, minus the electrical activity, the body is reasonably nondescript, reflecting the large diamagnetic contribution of the local water content. There are some notable exceptions, however. Farell et al. developed a SQUID magnetometer to non-invasively determine concentrations (as determined by magnetic susceptibility) in the liver. See, Farrell, D. E., Tripp, J. H., Zanzucchi, P. E., Harris, J. W., Brittenham, G. M. and Muir, W. A., “Magnetic Measurements of Iron Stores”, IEEE Trans. Magn. Mag-16, 818-82, incorporated herein by reference. This method has turned out to be very promising for diagnosing abnormal iron stores in the liver.

Clarke (Clarke J., Josephson Junction Detection, Science 184, 1235-1242 (1974), incorporated herein by reference) developed the first SQUID measuring device in 1974; twelve years after Josephson pointed out that supercurrent in a superconducting ring could tunnel through a small resistive barrier. This effect produces interference in the current wavefunction and hence current variation when the magnetic flux through the loop deviates from an integral number of quantized magnetic fluxoids. Measurement of magnetic field through the loop can therefore be measured against single quanta of magnetic flux. Since that time, radiofrequency (RF) SQUIDs have become the mainstays of commercial SQUID technology due to their stability. System design and the ambient fields in the vicinity of the probe determine noise levels. Since the inherent sensitivity (noise limit) is about 2×10⁻¹⁴T/\sqrt{Hz} (see, IImoniemi R. J., Williamson S. J., Kaufman L., Weinberg H. J. and Boyd A. D., “Method for Locating a Small Magnetic Object in the Human Body”, IEEE Transactions on Biomedical Engineering. Vol 35., No. 7, 1988, incorporated herein by reference) operational bandwidths (e.g., 0.1 Hz to 40 Hz) should allow for sensitivities as low as approximately 1.5×10⁻¹³ Tesla. System design and the ambient fields in the vicinity of the probe determine noise levels. Using gradiometer configuration pick-up coils noise levels on the order of 2×10⁻¹² T/m are readily achievable with good system design in an unscreened relatively low noise environment. See, “Magnetite Biomineralization and Magnetoreception in Organisms”, Ed. Kirschvink J. L., Jones, D.S. and MacFadden, B. J., Plenum Press (1985). A diagram which summarizes the sensitivities of some of the more common magnetometry techniques as a function of frequency is shown in FIG. 1. See, Malmivuo J. and R. Plonsey, “Biomagnetism: Principles and Applications of Bioelectric and Biomagnetic Fields”, Oxford United Press, 1995, incorporated herein by reference.

Because biomagnetic signals in the body are very small it was not until the advent of SQUID technology that practical measurements of these signals have been attainable. Since the advent of SQUID technology much effort has gone into imaging and mapping the biomagnetic signals produced by the brain and heart. SQUID helmets with large numbers of array elements (>100) have been produced to measure and map brain signals. See, Ahonen A. I., Hamalainen M. S., Kajola M. J., Knuutila J E T, Laine, P. P., Lounasmaa O. V., Parkkonen L. T., Simola J. T. and Tesche C. D., “122-channel SQUID Instrument for Investigating the Magnetic Signals from the Human Brain”, Physica Svcripta. Vol. T49, 198, 1993, incorporated herein by reference. Magnetocardiography (MCG) and Magneto-Encephalography (MEG) signals are time varying and produce changes in the magnetic flux outside the body. Sophisticated imaging models, based on the theory of reciprocity, have been developed using current distributions in current volume models to model the field produced by electrical currents circulating in a volume of tissue in the body. In principle, the theory of reciprocity (developed by Helmholtz 1853) leading to the principle of superposition tell us that the magnetic field generated at a detector by a volume of dipole moments is equivalent to the field produce by a sum over the individual dipole moments. This principle should be suitable for use in determining the position and size of the magnetized tumor with a background.

SQUID scanner systems have also been developed for use in Non-Destructive Evaluation (NDE) of materials. Limitations in the cost effectiveness and penetration depths have hindered the techniques commercial introduction and most SQUID NDE evaluation still takes place in university laboratories. SQUID scanners using an alternating current (AC) field (see, Bastuscheck C. M. and Williamson S. J., “Technique for Measuring the AC Susceptibility of Portions of the Human Body or Other Large Objects”, J. Appl. Phys 58(10) 1985, incorporated herein by reference) have been used to determine the magnetic susceptibility of human organs in the body. However, the AC field introduces a range of issues such as electronic stability, balancing, eddy current noise, etc. that need to be avoided. R. Ilmoniemi et al. developed a SQUID scanner which addresses may of these issues, but is intended to scan for a ferromagnetic inclusion (i.e., acupuncture needle) and hence their system has no inducing magnetic field. See, Ilmoniemi R. J., Williamson S. J., Kaufman L., Weinberg H. J. and Boyd A. D., “Method for Locating a Small Magnetic Object in the Human Body”, IEEE Transactions on Biomedical Engineering. Vol 35., No. 7, 1988. Other similar systems, used to measure iron stores in the liver, have also been described in the literature. See, Farrell, D. E., Tripp, J. H., Zanzucchi, P. E., Harris, J. W., Brittenham, G. M. and Muir, W. A., “Magnetic Measurements of Iron Stores In the Liver”, IEEE Trans. Magn. Mag-16, 818-82; and Paulson D. N., Fagly R. L., Toussaint R. M. and Fischer R., “Biomagnetic Susceptometer with SQUID Instrumentation”, IEEE Transactions on Magnetics, MAG27, 1990; both of which are incorporated herein by reference.

All of the SQUID magnetic scanners we are aware of to date use either liquid He or liquid N₂ to cool the SQUIDs and pickup coils in the system. This severely limits the geometry of the sensors to vertical (or near vertical) positioning above the target. Recently Quantum Design has introduced a new cryogenic refrigeration system which uses Joule Thompson cooling thereby eliminating the need for cryogenic fluids and opening the possibility of building scanners in horizontal or other more versatile geometries.

To date, static SQUID scanners have been used to map the electrical signals generated by the brain and heart. By static we mean that the sensors and the patient are rigid with respect to each other and the flux change through the sensors is induced by changing electrical activity in the organ. As of yet no continuous body scans of the resolution we are proposing has been performed. Accordingly, there is still a need for more specific, inexpensive, non-toxic, and non-invasive methods for detection of many types of cancer. Magnetic detection of tumor seeking molecules bearing superparamagnetic labels has the potential to become such a screening modality. The present invention satisfies those needs, as well as others, as will be more fully described herein.

### BRIEF SUMMARY OF THE INVENTION

The present invention generally comprises a magnetic body scanning method and apparatus for scanning all or part of the body for a magnetic signature of a cluster of ferromagnetic nanoparticles in relation to the diamagnetic signature of the body. Since there are no naturally occurring ferromagnetic particles in the body, the particles can be detected as they move through the body.

One aspect of the invention pertains to a method for targeting specific cells for imaging. In the preferred embodiment of the invention, a biological material, such as monoclonal antibodies, are attached to ferromagnetic nanoparticles with a bonding agent, and are then introduced into the body. The monoclonal antibodies seek out specific cells, such as cancer cells, and cause the ferromagnetic particles to cluster around those cells. When the cluster of ferromagnetic particles is sufficiently large, a net magnetic moment is created that can be detected with the body scanner. By selecting specific antibodies that target particular cells or tissue sites for imaging, and attaching those antibodies to ferromagnetic particles, a magnetic body scanner can be used to locate the ferromagnetic particles that have clustered around the target cells.

Another aspect of the invention pertains to a magnetic body scanner for locating the nanoparticles. In the preferred embodiment of the invention, the body scanner comprises a magnetic flux measuring device, such as a SQUID, Flux Gate Magnetometer or GMR magnetometer. As the body passes through the magnetometer, a line scan of the magnetic signature of the length of the body is produced, thus providing identification of the location of the clusters of ferromagnetic particles. In this way, the cancerous or other target cells can be located.

According to a further aspect of the invention, the body scanner comprises a table having an internal block that houses the SQUIDs that function as the sensors. A plurality of SQUIDS are used for the scanner to enhance resolution. To induce the magnetic field, each SQUID has an associated split coil, preferably of a superconducting type with the coil split into two windings, wherein the windings are counterwound in relation to each other. The rectangular block that houses the SQUIDs is preferably fairly narrow, but sufficiently wide to span beyond the full width of the patient's body. A conveyor or other device moves the patient over the block and through the coils to perform a line scan. As patient gets scanned across the table, the incoming signal produces a broad image of the body. When the ferromagnetic nanoparticles are scanned, a series of spikes is produced with amplitudes that are a function of the depth in the body. As a result, depth is added to the 2D line scan for 3D resolution that allows the location of the nanoparticles (e.g., tumor location) to be pinpointed. It will be appreciated that a significant difference between the present invention and, for example, conventional MRI is that the present invention senses the magnetic field that is induced and the resulting change in dipole moments due to the nanoparticles.

Another aspect of the invention is to detect and localize the magnetic field created by superparamagnetic particles localized on the tumor through phagocytosis or attached to tumor antigen-specific monoclonal antibodies targeted to occult human cancers. In recent years, the technology for covalently linking dextran-coated iron particles to monoclonal antibodies has been perfected. Such labeled antibodies have been shown to bind to specific cellular targets in vitro and in vivo. In vitro, iron-labeled antibodies have been used to highly purify specific rare cell types such as hematopoetic stem cells from a complex cell mixture. Iron-labeled antibodies can also be administered safely in vivo in humans and have been shown to localize at specific tumor targets as efficiently as standard radiolabeled antibodies.

A still further aspect of the invention is to use a SQUID to detect magnetically enhanced cancer tumors in the body leading to the development of a magnetic body scanner. The scanner envisioned would provide a rapid cost effective method for screening for many types of cancers.

Further aspects of the invention will be brought out in the following portions of the specification, wherein the detailed description is for the purpose of fully disclosing preferred embodiments of the invention without placing limitations thereon.

### BRIEF DESCRIPTION OF THE DRAWINGS

The invention will be more fully understood by reference to the following drawings which are for illustrative purposes only:

FIG. 1 is a diagram which summarizes the sensitivities of several common magnetometry techniques as a function of frequency.

FIG. 2 is a schematic diagram showing computer simulated body, tumor and scan geometry according to the present invention.

FIG. 3 is a schematic diagram of a SQUID scanner modeled in simulations according to the present invention.

FIG. 4 is a schematic diagram of a counterwound pickup coil in the scanner shown in FIG. 3.

FIG. 5 is a graph showing absolute magnetic field (single coil) at the scanner due to enhanced magnetization as a function of distance from the scanner.

FIG. 6 is a graph showing a line scan of magnetic field differential versus scan distance from a tumor located 5 cm from the central pickup coil of a SQUID sensor.

FIG. 7 is a graph showing signals from different pickup coils of a SQUID sensor from a tumor located 5 cm from the SQUID sensor where the signals are superimposed.

FIG. 8 is a graph showing a signal from a tumor located 10 cm from a SQUID Sensor.

FIG. 9 is a graph showing the differential magnetic field signal maximum as a function of distance form the SQUID scanner.

FIG. 10 is a graph showing the variation of absolute magnetization inflection point versus distance from the scanner for a 1-cm³ tumor.

FIG. 11 is a graph showing the differential magnetic field signal as a function of the applied induction field calculated for a 1-cm³ tumor located 10 cm from the SQUID scanner.

FIG. 12 is a graph showing a differential magnetic field scan (output from first order pickup coil subtraction) as a function of scan distance for a 1 cm³ tumor located 100 mm from the y-axis and located at a distance of 5 cm from the central scan point and a depth of 4 cm below the surface of the diamagnetic ellipsoidal background.

FIG. 13 is a graph showing a line scan for a tumor at 5 cm plus virtual organ background plus diamagnetic background for an ellipsoidal background.

FIG. 14 is a schematic diagram of a SQUID Dewar, scanner, and proximity transport system according to the present invention.

FIG. 15 is a schematic diagram of an interchangeable solenoid and pickup coil platform according to the present invention.

FIG. 16 is a top plan schematic view of a transport chamber for use with the present invention.

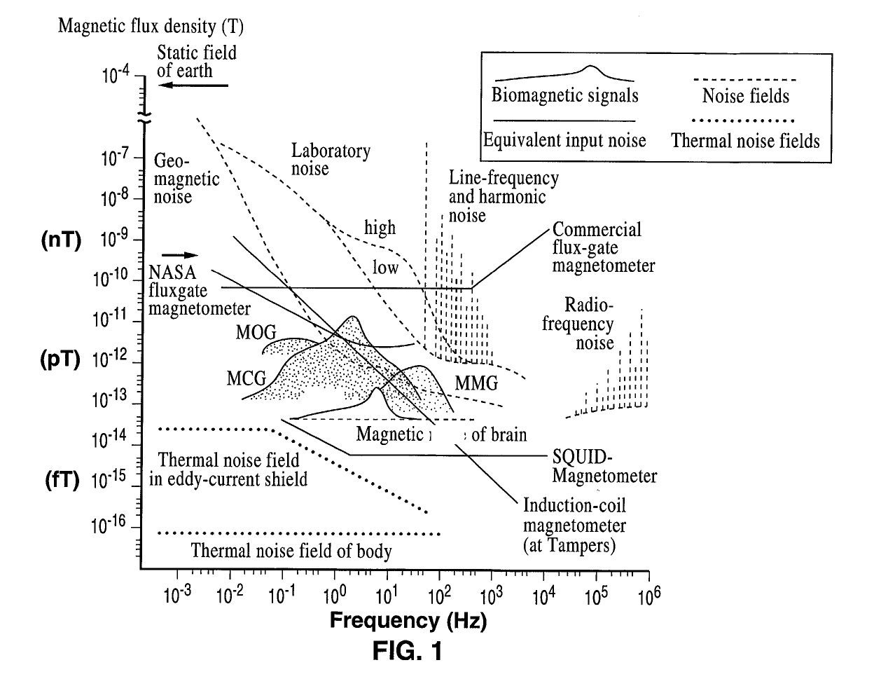

### DETAILED DESCRIPTION OF THE INVENTION

A system for measuring the signature of a localized superparamagnetic particles within the body according to the present invention generally comprises means for generating an induction field, means for inducing a magnetization induced flux change, and means for measuring a magnetization flux change as described herein. Such a system should be both stable and sensitive.

While the physical parameters to be measured with such as system are necessarily design dependent, the system can be simulated as described below and the problems inherent in the design of this type of measurement can be solved in a straightforward manner. Development of a magnetic body scanner according to the present invention involves simulating the physical parameters involved in order to obtain an accurate assessment of signal of the superparamagnetic inclusion, signal of diamagnetic background of the body, and optimization of the geometry of the measuring device.

The magnetic nanoparticles employed are preferably superparamagnetic (SP) particles. In Magnetite and Maghemite this phase occurs for particle sizes less then 25 nm. Similar to single domain (SD) particles, SP particles have a net spontaneous magnetic moment; however, unlike SD particles the strength on the volume dependent anisotropy constant is small enough so that it can be overcome by thermal fluctuations. In this state, the particle magnetization instantaneously aligns in the magnetic field. For example, we have performed AC-susceptibility measurements of 20 nm sized Maghemite particles (e.g., Miltenyi Microbeads in solution), in a Quantum Design SQUID Magnetometer, and have found effectively perfect alignment of the magnetization (constant in-phase susceptibility and zero out-of-phase susceptibility) with the field for field frequencies up to 1 kHz. This is well above the preferred frequency range measured with the present invention and we can neglect any sample magnetization time dependences (remanences).

### EXAMPLE 1

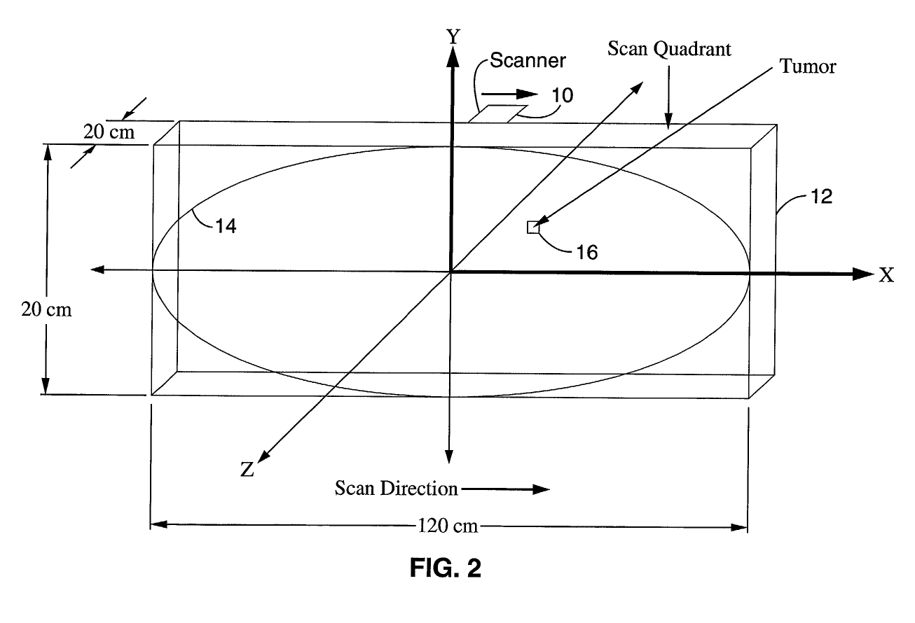

Initial simulations were performed to identify theoretical limitations and determine the physical parameters under which a SQUID coupled sensing device could obtain a signal from superparamagnetic inclusion located in the body. To do this, we developed a brute force three-dimensional simulation of the body, superparamagnetic inclusion, applied magnetic field, and SQUID sensing coils. By necessity, a scanner design must be chosen and a particular protocol simulated. Referring to FIG. 2, in our simulations we chose a scanner 10 which incorporates a DC superconducting induction field (low noise and highly stable) and uses motion of the patient to produce a flux change in the pick up coils. This design is simple, cost effective and flexible.

The phantom torso (body) in our simulations was modeled as an ellipsoid filled with water. The Molar Diamagnetic susceptibility of water is −13×10⁻⁶ (emu). This approximation was used to determine the maximum absolute contribution of a large slowly varying diamagnetic background (of approximate torso dimensions) with rapidly varying edges. There are clearly limitations on the information that can be obtained from this simple model, but the model is useful for determining theoretical limits of the technique.

A three dimensional rectangle 12 was formed with dimensions of 120 cm in length, 20 cm in width and 20 cm in thickness. The dimensions of the rectangle were then divided into mm³ cubes, with the magnetization from each cube contributing to the measured signal. The torso was modeled in the rectangular box as an elongated ellipsoid 14 with the same maximum dimensions as the rectangle 12.

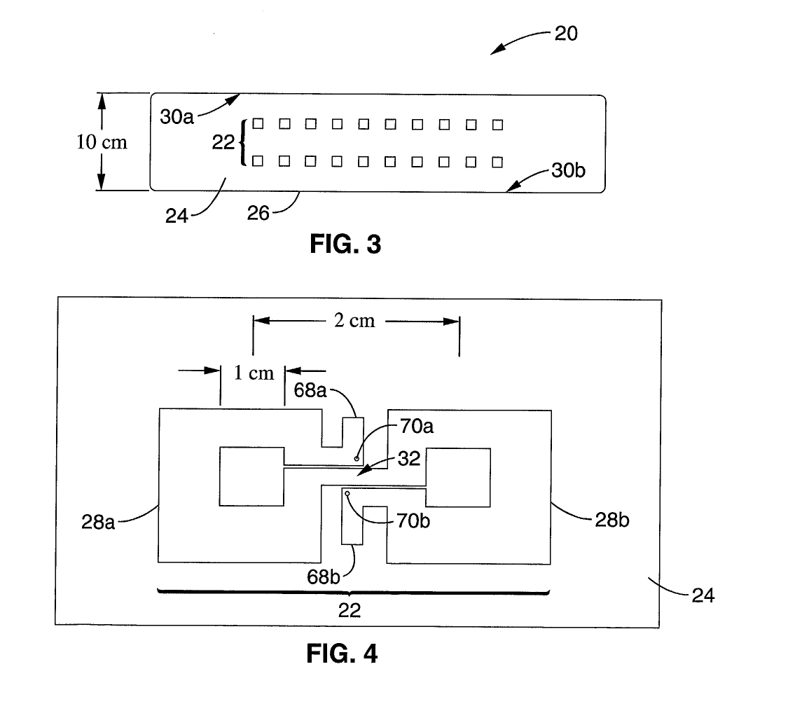

The scan covered the upper positive quadrant of the three dimensional rectangle and hence the upper quadrant of the ellipsoid. The rectangle and ellipsoid were shifted by 10 cm from the X-Y plane for mathematical simplicity (all positions in this quadrant are positive definite). The pickup coils, located in the scanner 10, were located 1 cm above the top of the rectangle. The magnetic field was in a “racetrack” configuration centered around the scanner platform as shown in FIG. 3.

During the summing of the magnetic field contributions of the magnetization, it was found that cubes located outside the ellipsoid produced no contribution to the field at the sensing coils. On the other hand, cubes located inside the ellipsoid had a diamagnetic response to the applied field and produced a corresponding contribution to the field at the SQUID sensing coil.

### EXAMPLE 2

The tumor 16 was modeled as a paramagnetic inclusion located at various positions within the ellipsoid 14. In the mm representation, the tumor with magnetic particles was represented as a 1 cm×1 cm×1 cm cube containing 1000 mm³ cubes. These “tumor” cubes had both a diamagnetic contribution and a paramagnetic contribution due to the magnetic nanoparticles. The paramagnetic contribution was calculated using the parameters given in Shen et. al., who studied the behavior of magnetic nanoparticles uptake by mouse brain tumors as a contrast agent for MRI. See, Shen et al., Monocrystalline Iron Oxide Nanocompounds (MIONS) Physicochemical Properties, Magn. Reson. Med 31, 599-604 (1994). Using a value of (100 ng of iron)/(1 million tumor cells) and taking the average diameter of a tumor cell to be 20 μm, we estimated that a 1 cm³ tumor to contain 12.5 μg of Fe. From the graph of the magnetization vs. field in Shen et al. we took the magnetic susceptibility for fields less then 2 kG to be 2.2×10⁻² emu/gm (Fe).

### EXAMPLE 3

The magnetic field was modeled as a “racetrack” geometry extending across the width of the body. FIG. 3 is top plan schematic illustration of a SQUID scanner 20 modeled in simulations. The scanner comprises an array of ten first order gradiometrer pickup coils 22 located to the interior of and on the same platform 24 as a superconducting solenoid 26. FIG. 4 shows the configuration of one of the pickup coils 22. Preferably, the centers of loops 28a, 28b that make up an individual pickup coil 22 are separated by 2 cm as shown. In the simulations, the long sides 30a, 30b of the “racetrack” shaped solenoid are separated by 10 cm. The value of the magnetic field is the field generated at the midpoint of a set of coils of a pickup coil. This is also defined as the scan point 32.

### EXAMPLE 4

Neglecting end effects (extending the magnet well past the width of the body), the magnetic field was modeled as a contribution from the two wires separated by 10 cm, each wire located 5 cm on opposite sides of the scan point. The magnetic field located at a distance r from the wire has a magnitude equal to B(r)=C/r where C is a constant determined by the magnetic field at 1 cm. This field was taken to be 5000 G, producing a total magnetic field of 2 kG at the central scan point. The field is a vector quantity radiating tangentially from a circle or radius r centered on the wire. The field due to the second wire circulates in the opposite direction giving a significant cancellation of the x components near the vertical line passing through the scan point. The y-components of the magnetic fields located near the same vertical line add, producing strong vertical polarization of the diamagnetic ellipsoid and the magnetically enhanced tumor. By knowing the direction of the magnetic field and magnetic susceptibility at any point in the matrix, the magnetization vector can be calculated. Treating the magnetization of a mm³ cube as a magnetic dipole we calculated the magnetic field produced by the sample magnetization at the position of the pick up coils.

### EXAMPLE 5

To minimize ambient noise and the background signal, the pickup coils were modeled in a planar first-order gradiometer configuration (see, Ketchen M. B., “Design of Improved Integrated Thin-Film Planar DC SQUID Gradiometers”, J.Appl. Phys, 58, 11 1985, incorporated herein by reference) with each of the counter wound pickup coils having an area of 1 cm² and each coil located along the length-axis with the center of the coils displaced 2 cm on either side of the scan point. The magnetic field signal due to the inclusion and/or a background cube was calculated at a point at the center of each coil. The coils were assumed to be 1 cm² each and the field was assumed to be uniform over the area of the coil.

### EXAMPLE 6

The scan covered the upper positive quadrant of the three dimensional rectangle and hence the upper quadrant of the ellipsoid. The rectangle and ellipsoid were shifted by 10 cm from the X-Y plane to allow for a scan across the full width of the body. In general the pickup coils were placed 1 cm from the top of the ellipsoid. A scan at any one scan point included a scan volume of 100 mm along the length, 200 mm along the width and 100 mm of thickness and the magnetic field was calculated at each of the two counterwound pickup coils for each of the ten scan elements. The 100 mm length of the scan volume had the effect of clipping the generated signal at distances greater then or equal to 5 cm from the tumor along the length axis. As the scanner was moved the length of the body, six hundred of these scan volumes were included in the total scan. We estimated that the total ten SQUID simulation included approximately 1×10¹¹ calculations and takes approximately 6.5 hours on a 960 MHz Pentium III PC. Even so the mm³ grain size appeared as rapid jumps in the background contribution as the mm³ grains were limited by the smooth ellipsoidal function.

### EXAMPLE 7

Exploratory phase measurement with phantom tumors can be performed in an electromagnetically shielded screened room. By developing experiments in a screened room, much of the electromagnetic noise is eliminated which may otherwise hinder the ability to accurately determine the signal to noise ratio of the design and characterize the signal induced by the phantom tumors. A commercially built (Lindgren and Associates Inc.) screened room is covered in bronze mesh providing 120 dB of screening above 10 kHz and greater then 30 dB of magnetic screening. With such a screen room, we expect to achieve at least 10 dB reduction in electric field noise and greater then 5 dB noise reduction in field due to magnetic dipoles in the lower frequency range of interest (0.1 Hz to 40 Hz). Since we are performing an effectively DC experiment, we do not expect to induce a significant noise contribution from the screening material. The interior dimensions are 3 m×3 m×2.5 m, leading to lowest order waveguide modes above 100 MHz well above our region of interest. Several modifications need to be made to the room. One noise issue is the noise generated by the control and data acquisition computer. This can be addressed by placing the scanner and computer at opposite ends of the room, by placing the computer in a secondary screened volume, or by moving the computer completely outside of the screened room and filtering the computer lines going into the screened room.

### EXAMPLE 8

We have performed a set of computer simulations to determine the feasibility of measuring and mapping the magnetic fields produced by superparamagnetic nanoparticles which have been aligned by an external magnetic field. Simulations were constructed using values for the concentration of iron in nanoparticles associated with tumors in an in vivo mouse model. An induction field polarizes the nanoparticles magnetic moment. As a patient is transported past the scanner, the aligned magnetic moments produce magnetic flux changes in a planar first order gradiometer coil. The generated signals by a 1 cm³ tumor, at a distance of 10 cm, are of sufficient strength to be detected with a DC SQUID amplifier. These simulations allow determination of physical parameters important to the development of this type of magnetic scanning technology and the simulations demonstrate the feasibility of using SQUID magnetometry for in vivo detection of magnetic labels targeted to specific structures.

### EXAMPLE 9

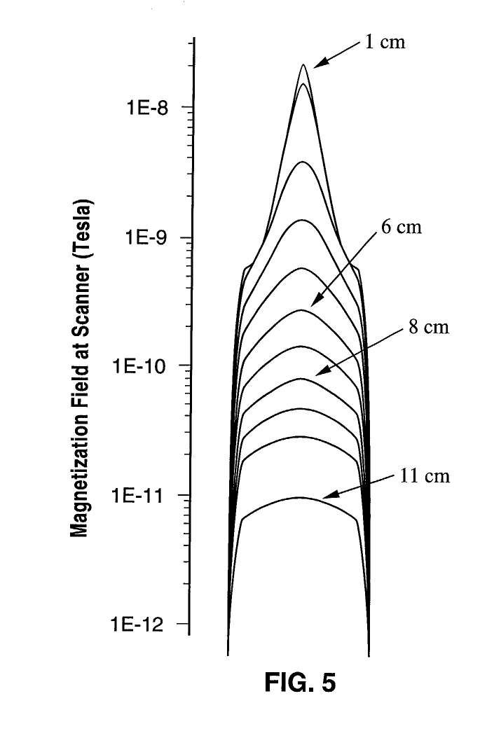

FIG. 5 shows the absolute magnetic field generated by a 1 cm³ tumor at various distances from the SQUID scanner, ranging from 1 cm to 10 cm. Edge effects of the signal are due to a finite scan width and accentuated by the log scale. The magnetic field applied was 0.2 T (2000 G) at the scanner. In this range, it appears that the signals are well above detection limits but in practical applications detection limits of small signals are generally determined by the ambient magnetic noise. Noise reductions of two to four orders of magnitude can be achieved with a well-balanced gradiometer configuration for the pick-up coils. While the differential magnetic field detected by gradiometer configurations is smaller then the absolute magnetic field, this difference is more then compensated for by the reduction (cancellation) in ambient noise. The data presented below are from a first gradiometer pick-up coil with center to center coil distance of 4 cm. As such the signal measured is a differential magnetic field.

### EXAMPLE 10

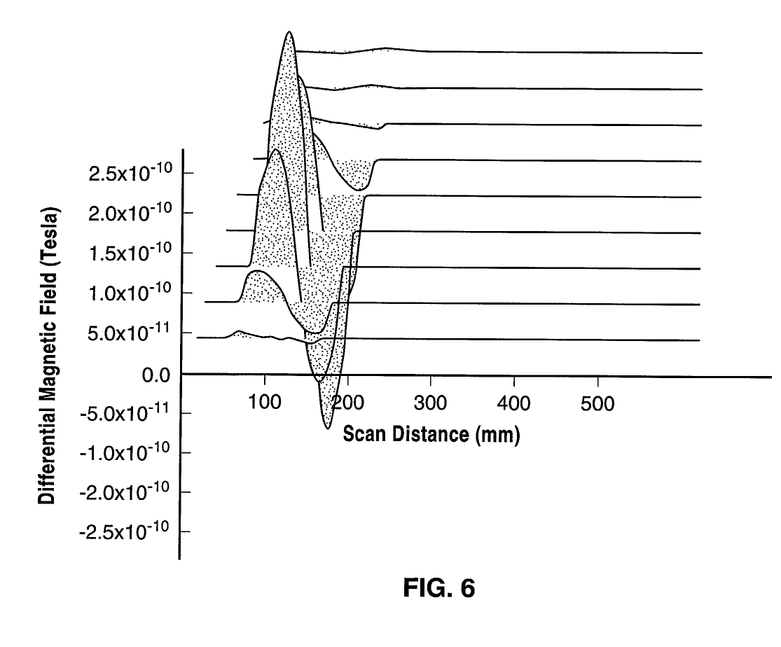

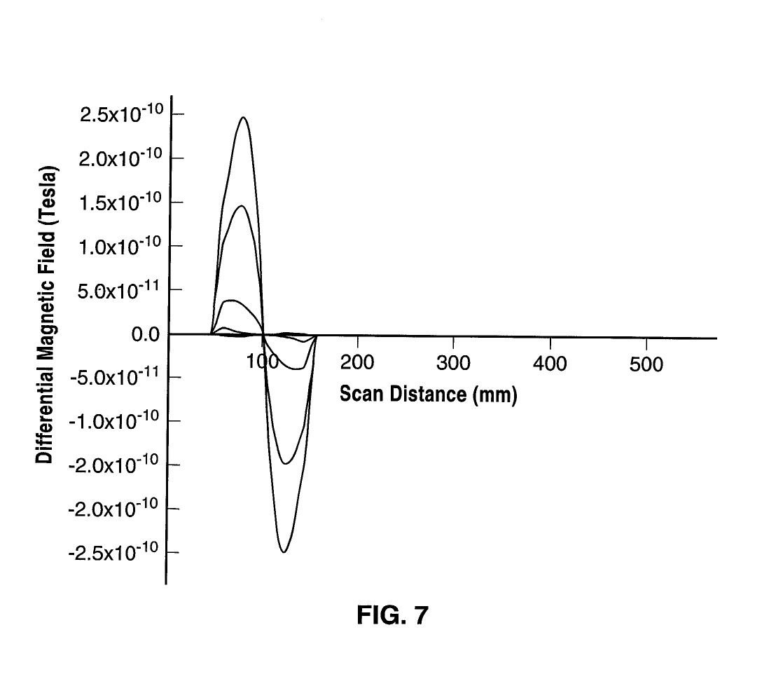

FIG. 6 and FIG. 7 show the signals generated from the tumor located 5 cm from the central pickup coil. FIG. 6 shows the spatial distribution of the signals at the various pickup coils in the scanner for the following parameters: no background or continuous background (rectangular box filled with water); tumor is located at x=10 cm (100 mm) y=6 cm (5 cm from pickup coils) and z=10 cm (center of the ellipsoid); scan produced by 10 SQUID scanner; and maximum amplitude scan located at scanner with coordinates x=10 cm, y=11 cm and z=10 cm. In FIG. 7, the signals from the different pickup coils in the scanner are superimposed to aid the eye in the reading of the actual signals.

As the scanner moves across the length of the scan rectangle the pickup coil on the near side begins to pickup the signal. The signal then maximizes close to the point where the pickup coil is positioned vertically over the tumor. The signal then goes to zero when the scan point is directly over the tumor and then goes negative as the other counterwound coil passes over the tumor. The maximum signal is close to 1×10⁻⁹ Tesla well within the limits of this type of scanner technology. The key to using this technology is that the pickup coils will need to be able to sense at 10 cm.

### EXAMPLE 11

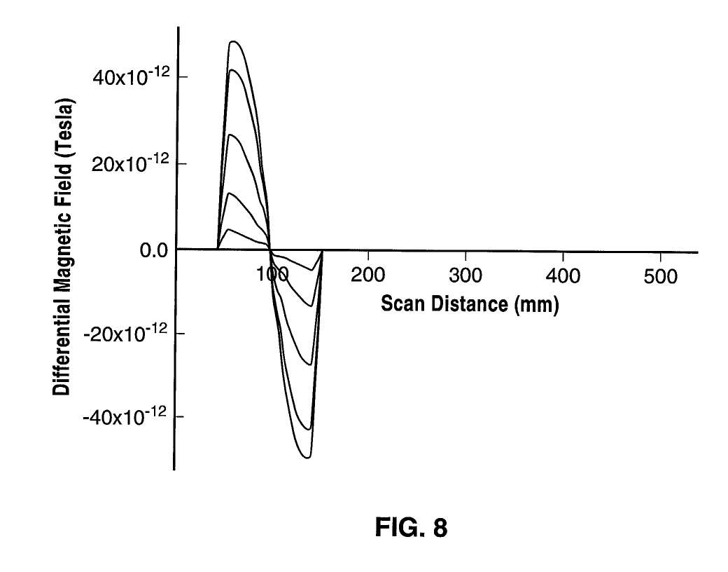

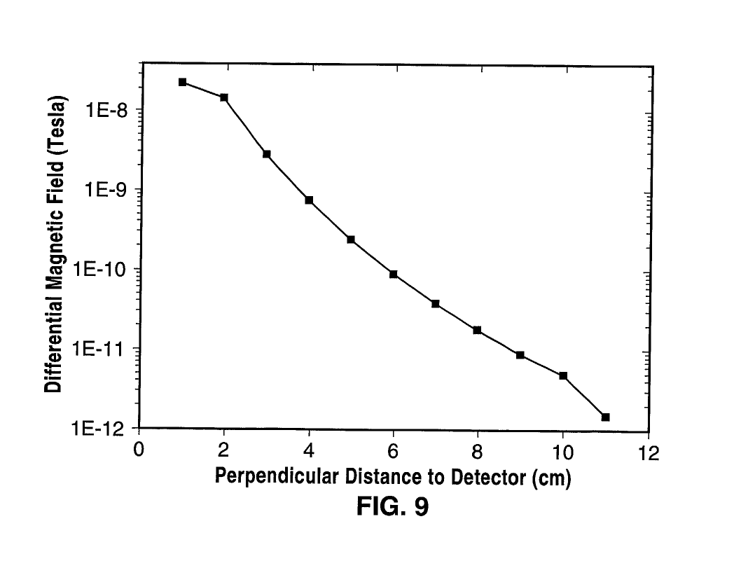

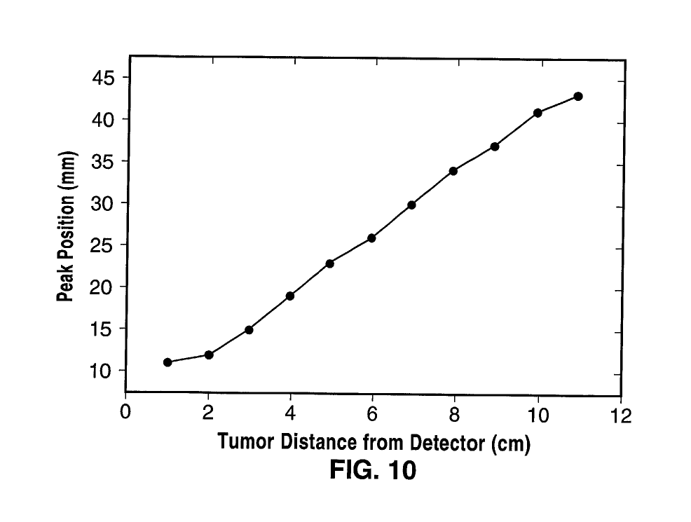

Approximating the thickness of the body to be 20 cm (for a patient lying on a flat table), a complete body scan can be accomplished by having the patient scanned both over the front and the back of the body. FIG. 8 shows the raw signal for the tumor located 10 cm from the pickup coils. FIG. 9 is a plot of the maximum differential signal (signal from the pickup coil) from the tumor as a function of distance of the tumor from the scan point of the detector. The theoretical signal produced by the first order gradiometer pickup coils for an isolated 1 cm³ tumor drops off rapidly as a function of distance from the scanner but is still theoretically within SQUID resolution even at a distance of 11 cm from the scanner.

The signal from the tumor located at 10 cm from the pickup coil is interesting for two reasons. First the magnitude of the signal is still larger then the technique resolution in an unscreened environment. However the signal to noise ratio is not adequate for realistic and reproducible detection considering an expected ambient noise of order 10⁻¹² Tesla. The second interesting feature is that the structure of the signal is different from the 5 cm scan. This adds a second dimension to the analysis opening the possibility of obtaining depth information from the form of the signal.

### EXAMPLE 12

FIG. 10 shows the variation of absolute magnetization inflection point versus distance from scanner. The inflection point corresponds to a peak in the in the Differential Magnetic Field signal. While the signal is an order of magnitude greater than a minimal signal, a signal to noise ratio of 10 to 1 is not optimal strongly enhancing the need to increase the magnetization on the tumor. This can be achieved by increasing the applied magnetic at 10 cm, increasing the number of magnetic particles per tumor, finding particles with a larger magnetic susceptibility, or a combination of all three.

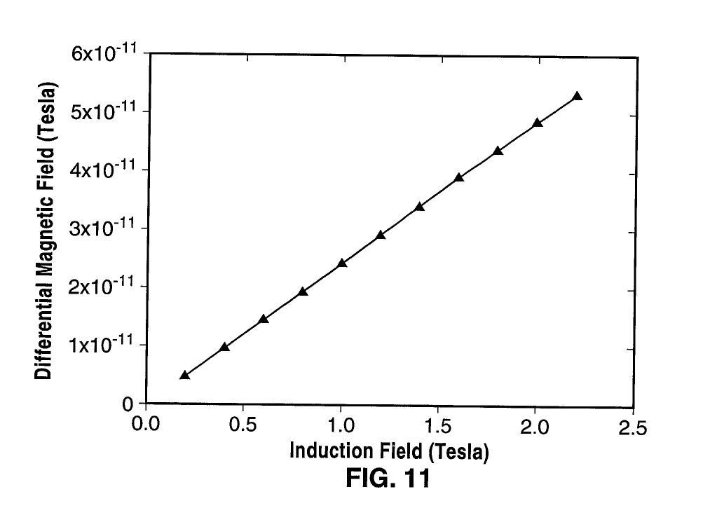

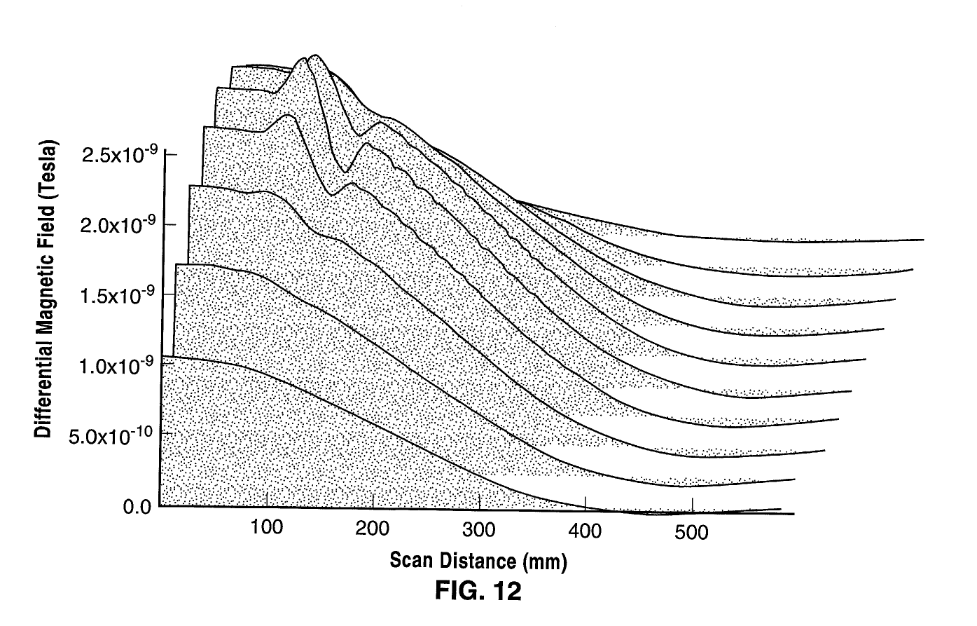

FIG. 11 is a plot of the differential magnetization field at the SQUID scanner as a function of the applied magnetic field as defined above for this geometry. The signal being amplified by the SQUID is a linear function of the applied field. For an applied field of 2-Tesla the actual induction field at the tumor 10 cm from the pickup coils is 1800 Gauss, still within the linear region of the susceptibility. Therefore with application of a 2-Tesla field, the signal can be increased by an order of magnitude. An increase in the DC Field will however, make the system even more sensitive to vibrational noise. We next simulated the effects of the diamagnetic background of the body. FIG. 12 shows the differential magnetic field scan (output from first order pickup coil subtraction) as a function of scan distance for a 1 cm³ tumor located 100 mm from the y-axis and located at a distance of 5 cm from the central scan point and a depth of 4 cm below the surface of the diamagnetic ellipsoidal background. Simulated signal generated by 10 SQUID Scanners scanning the width of the ellipsoid. The noise on the background is an artifact of discontinuities caused by the mm³ cubes bumping up against the boundary of the continuous ellipsoid. The magnetic induction field applied was 0.2 T (2000 G) at the scanner. The signal from the tumor at a depth of 5 cm depth can easily be observed against the diamagnetic background signal. At larger depths (>6 cm distance for scanner) the signal is difficult to discern due to the large artificial noise component in the simulation. It can be observed that the tumor at 5 cm has a signal approximately equal to the maximum signal of the diamagnetic background and can therefore be resolved. It can also be observed that the background has a noise associated with it, which appears as ripples. These “ripples” are an artifact of the algorithm. The body is simulated as an ellipsoidal function. When the mm sized cubes, which are being summed over, bump up against the continuous ellipsoidal function we get a staircase like pattern in the cubes producing a discontinuous signal. This artificial noise is more then an order of magnitude larger then the signal of the tumor at 10 cm and therefore the tumor cannot be resolved against this background.

### EXAMPLE 13

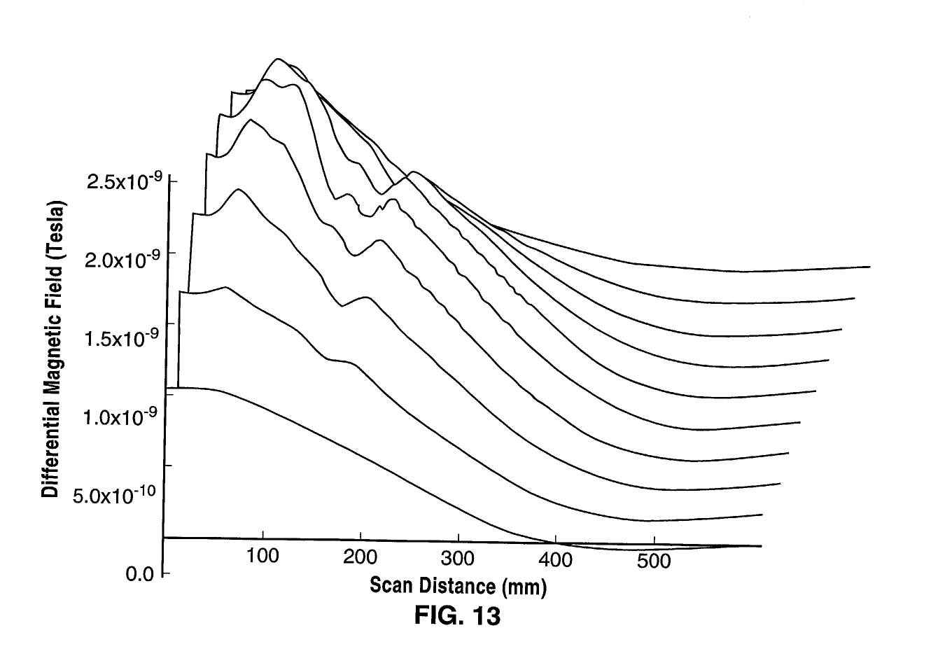

To further follow up on the discussion of background signal, we investigated the effects of a uniform background signal and a tumor located in say a large organ. Moore et al. who studied the uptake of Long Circulating Dextran-coated Iron Oxide Nanoparticles (LCDIO) by 9L gliosarcoma brain tumors in a rodent model raise this issue. See, Moore A., Marecos E., Bogdanov A. and R. Weissleder, “Timoral Distribution of Long-Circulating Dextran-coated Iron-Oxide Nanoparticles in a Rodent Model”, Radiology 2000; 214:568-574. These vary small particles show minimized uptake by the reticuloendothelial system and uptake by tumor vasculature. In this sense these particles are not specifically targeted to the tumor with a targeting molecule such as a monoclonal antibody. In this study they found that tumor uptake by the brain tumor was approximately 0.11% of injected dose. They also found that the surrounding healthy brain tissue LCDIO concentration was approximately 10% of the tumors concentration. The effect of this type of background signal was investigated in FIG. 13. A fictitious organ was modeled around a tumor that is located 5 cm from the scanner. The organ was modeled as a rectangular box with thickness 2.5 cm on either side of the tumor, width 5 cm on either side of the tumor and length 5 cm on either side of the tumor in the direction of the scan length. The organ was given an Iron Oxide concentration of 1% of the tumor concentration. The diamagnetic background of the body was included. The scan in FIG. 13 shows that a 1% organ background produces a signal comparable to the signal due to the tumor. At 1% the signal due to the tumor is still resolvable. Simulations were also done with a 10% background tumor. In these simulations the background completely overwhelmed the tumor signal. This provides limits on contributions due to surrounding tissue. On the other hand for intravenously administered targeted Iron Oxide particles conjugated with monoclonal antibodies it was found that tissue surrounding the tumor had “modest” uptake but no evidence of the presence of monoclonal antibodies. This suggests that well constructed magnetic label-target specific vector conjugate will provide the best system for maximizing tumor uptake while minimizing background.

In terms of the physical parameters analyzed in this simple model, values of magnetization in realistic magnetic induction fields are measurable with SQUID technology. Theoretical signal to noise ratios of between one hundred and eight hundred are predicted for an 1 cm³ tumor located 6 cm to 8 cm from the scanner, in a reasonably quiet environment. The diamagnetic signals from the body volume have signals on the same order as the tumor at 5 cm. While it may be possible to eliminate much of the background signal by surrounding the body with water the volume contribution of organs, bones etc. must also be accounted for. We also analyzed a 1 cm³ tumor at 5 cm within a simulated organ of thickness, width and length, 5 cm×10 cm×10 cm consisting of a diamagnetic water concentration and a 1% of tumor concentration superparamagnetic contribution. The organ contribution was as large as the tumor signal suggesting the need for efficient targeting.

### EXAMPLE 14

There are several variables that can be potentially modified to enhance the signals generated using this technique. We focused on the signal due to a paramagnetic inclusion located 10 cm from the detector. The signal is directly proportional to the amount of Fe in the tumor. The most promising method for enhancing the signal is optimization of the nanoparticles magnetization and size. While larger fields may be employed to increase the tumor signal, larger fields will also increase the background signal and the inherent noise in the system.

Implementation

Theoretically the magnitude of the signal of a 1 cm³ tumor at distances of 10, 11 or even 12 cm from the scanner (pickup coils) are accessible by SQUID technology in a low noise environment. To achieve this level of sensitivity, the configuration and spacing of the pickup coils should be optimized. In this regard, there are two factors that come in to play in consideration of the pickup coils. The first factor is to maximize the magnetic sensitivity by varying the pickup coil configuration, (i.e. dipole loop, 1^{st} order gradiometer, second order gradiometer etc.). The second factor is optimization of the spatial configuration of multiple pickup coil-SQUID system to maximize spatial resolution of the entire scanner.

Methods for significantly reducing the background include surrounding the body with water to produce a more uniform background and eliminate contributions from air pockets and edge effects. It is clear from the simulations that measurement of the localized moment will require the most sensitive type of SQUID Magnetometry; namely DC SQUID Magnetometry.

It will be appreciated that the theory of imaging magnetic sources at a distance from a scanner is quite well developed. A scanner that comprises scanning elements located across the width and scanned lengthways over the length of the body produces at minimum enough information for a two dimensional magnetic image. Correlations of signal structure with depth have shown evidence for identifying the depth of the tumor and hence giving a third dimension of information.

### EXAMPLE 15

In a preferred embodiment of the invention, a Model 601 LTS DC SQUID Scanner available from Tristan Technologies Inc. of San Diego is modified as described below. Tristan currently builds SQUID Scanner systems for measuring hepatic liver stores and for Magnetocardiography. As far as we are aware, Tristan Technologies Inc. is the only company that produces commercially available DC SQUID scanners. Modifying a commercial SQUID scanning device built for design flexibility eliminates many of the technical issues involved with setting up a SQUID system and allows for tapping into the expertise of several experts in this field. With 1 cm pickup coils Tristan reports that sensitivities approaching 10 fT per square root hertz are possible. Some of the technological difficulties involved with building a useful SQUID scanner include minimization of the dewar wall between the sensor and target, rigidity of field coils with respect to the pickup coils and electronics design. We preferably use a He cooled cryostat and the pickup coils will be positioned above the target as require by this type of cryostat. Tristan currently produces a Dewar that at the scan face goes from 4.2K to 300K in <5 mm. This feature is essential as the simulations show the signal decreases rapidly as a function of distance.

The SQUID signal is preferably filtered and processed through an analog-to-digital converter (ADC) connected to a personal computer (PC) or the like. The PC preferably includes software to control both a transport mechanism and the data acquisition. By sampling data at a reasonably fast rate compared to the transport velocity signal, averaging can be employed to improve the signal to noise ratio. The final array of scan data will have a spatial resolution of greater then 1 mm. The line scan is preferably stored as a linear array, as a function of scan distance.

### EXAMPLE 16

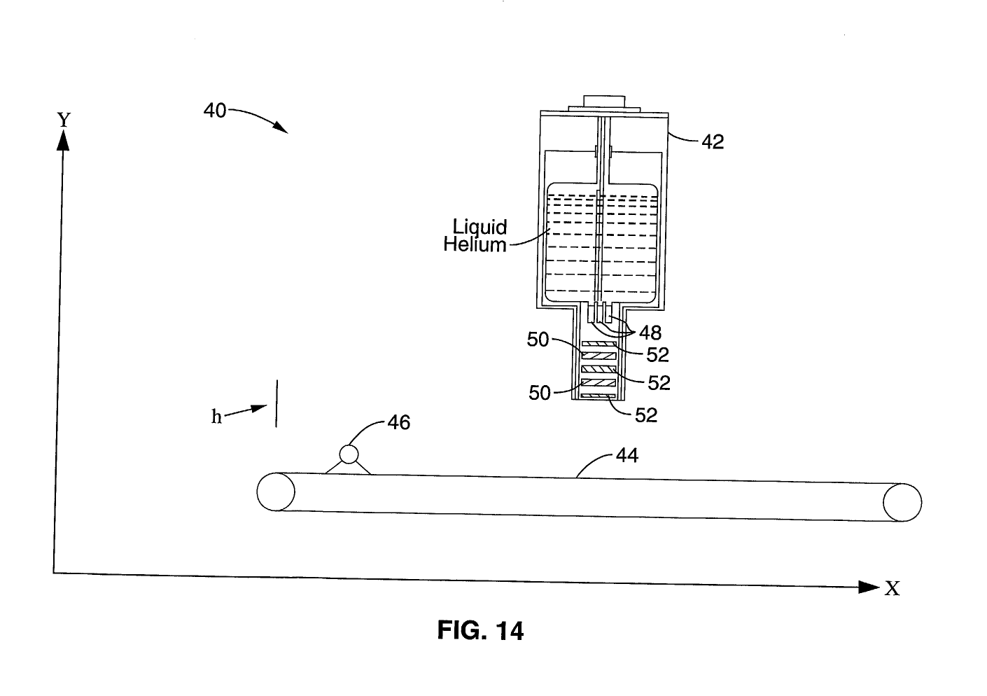

FIG. 14 schematically shows the configuration of a SQUID Dewar, scanner, and proximity transport system 40 configured for use in the present invention. The liquid He Dewar 42 is preferably fixed in an aluminum collar (not shown) located near the top of the Dewar. The collar is preferably supported by A-frame aluminum legs (not shown). The transport device 44, such as a transport table/belt or the like, conveys a sample 46 past the SQUID sensors 48 along the x-axis as shown. The SQUID sensors are positioned above the sample at a height h. In the embodiment illustrated, the superconducting magnet coils 50 and second derivative gradiometer detection coil 52 are shown for reference.

The transport device 44 is preferably located between, but not in contact with, the A-frame legs. Both the stand (not shown) for the Dewar and the transport table should be independently bolted to a solid stable floor structure (e.g., concrete) under the screen room (not shown). Low frequency vibrational damping can be added as required. Screening preferably will be accomplished at these floor contact sites by bolting through an eighth inch copper plate (not shown). Analog and stepping motors should be electromagnetically screened.

Note that scanners built by Tristan use wire wound pickup coils with the counterwound coils wound at different positions along the y-axis as shown in FIG. 14. While a second-order gradiometer geometry gives better noise cancellation and is preferred, first-order gradiometer coils are much less sensitive as a function of distance.

Referring again to FIG. 4, the pickup coils are preferably fabricated in a planar geometry (without integrated SQUID) using thin film technology and optical lithography techniques. These techniques allow for minimizing area differences between the coils down to the μm² scale. The pickup coils are preferably deposited on Si as a 500 nm film of Nb and patterned as shown in FIG. 4. This is a simple design, requiring only a single deposition and a single lithographic step. Using optical lithography and a chromium mask, estimates of pickup coil balance of greater than one ppm are achievable. Most of the balance error will come from the attached leads. This can be minimized by ultrasonically “drilling” two holes in the Si substrate. NbTi wire fed through the holes can be attached to the film pad by ultrasonic bonding. It will be appreciated that these pickup chips can be fabricated with various characteristics. For example, the chip shown in FIG. 4 will have high signal resolution but low spatial resolution. An alternative embodiment with dimensions approximately a factor of ten smaller than the chip of FIG. 4 will have good spatial resolution but less signal resolution due to the smaller pickup coil area. This smaller chip will also have better signal to noise ratio and better background subtraction.

### EXAMPLE 17

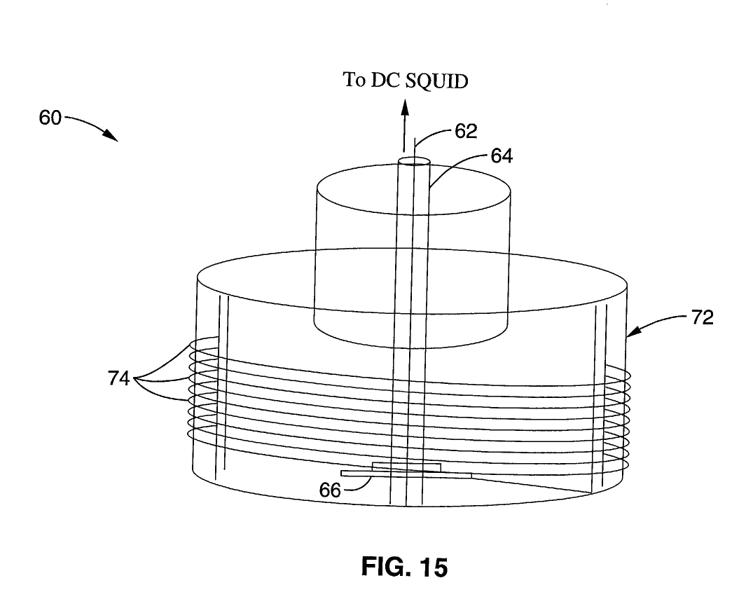

FIG. 15 schematically shows an interchangeable solenoid and pickup coil platform 60 according to the present invention. The NbTi leads 62 are twisted and epoxied to a G10 rod 64 extending from the back of the pickup coil chip 66 (e.g., chip 22 shown in FIG. 3 and FIG. 4) up to the entrance to the DC SQUID. The leads ends attached to the pickup coil pads 68a, 68b will thread through the holes 70a, 70b in the Si and converge, epoxied to the backside, where twining begins. To maximize vibrational stability, all components of the platform will be embedded in stycast epoxy (e.g., G10). Thus each pickup coil will have its own dedicated G10 platform 72 and solenoid 74.

Preferably, the magnetic field solenoid 74 is fabricated from NbTi (52%/48%) wire. The wire preferably has a diameter of 2.8×10⁻³ cm. In order to produce a 1T field (at the scan point) at 5A current approximately four layers of windings are required. Upon completion of winding, the solenoid is embedded in the stycast epoxy and a persistent switch is constructed at the top of the platform. The magnet can be powered by any conventional power supply.

Referring again to FIG. 14, the transport device 44 for a human body scanner should be designed to minimize magnetic and vibrational contributions. The transport device preferably comprises four sections, all isolated from contact with the scanner as discussed above. Two sections of the transport device effectively comprise a table on either side of the scanner. Two physically independent but electrically connected rotating belts on each table will provide the transport mechanism. The proximity of the belts to the scanner obviate the need for care to be taken to only use belt materials with small and small homogenous magnetic susceptibilities as would be the case with phantom samples. The table preferably have sides approximately 30 cm high to help support the sides of the body chamber.

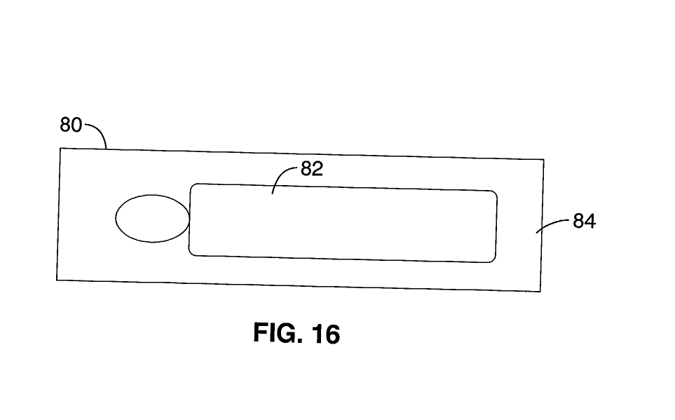

Referring to FIG. 16, the chamber 80 that the patient will be transported in preferably comprises a thin walled flexible plastic. The transport chamber preferably has walls approximately 20 cm high and sealed at the top and the bottom. The chamber has a foam body cavity 82 in which the human body is placed for scanning, and the chamber preferably comprises water filled foam 84 to decrease the background signal of the diamagnetic contribution of the body.

Transport velocities preferably range from approximately 2 cm/sec to approximately 20 cm/second. This will provide a comfortable scan speed for the patient and allow for rapid scanning taking approximately 10 s to 20 s per full body scan. This speed will also provide a magnetization change through the pick up coils at large enough frequency to minimize the low frequency noise inherent in SQUIDs.

Stepping motors used for driving the device should have both stepping and analog modes for optimization of the transport technique. Standard magnetometers that operate on the principle of Faraday's law produce a signal that is proportional to the time derivative of the change in magnetic flux (faster flux change gives a larger signal). The superconducting pickup coil loop integrates the signal making rapid scanning unnecessary. Instead, scanning rates are determined by optimization of SQUID signal bandwidth, patient comfort and efficiency. It may also be necessary to cycle all or a small part of the patient over the scanner to average the signal and increase signal to noise ratio.

Although the description above contains many specificities, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A method for locating cells in the body, comprising:
introducing a plurality of cell targeting ferromagnetic nanoparticles into the body; and
scanning the body for a magnetic signature of a cluster of the ferromagnetic nanoparticles in relation to the diamagnetic signature of the body.

2. A method as recited in claim 1, further comprising attaching a biological material to a plurality of said nanoparticles.

3. A method as recited in claim 2, wherein said biological material comprises a monoclonal antibody.

4. A method as recited in claim 2, wherein said biological material is attached to said nanoparticles with a bonding agent.

5. A method as recited in claim 2, wherein said biological material seeks out target cells in the body, such as cancer cells.

6. A method as recited in claim 5, wherein said ferromagnetic nanoparticles cluster around said cells.

7. A method as recited in claim 6, wherein said clustering causes a net magnetic moment.

8. A method for locating cells in the body, comprising:
attaching a biological material to ferromagnetic nanoparticles with a bonding agent;
introducing said cell targeting nanoparticles into the body;
wherein said biological material seeks out target cells in the body and causes said ferromagnetic nanoparticles cluster around said cells;
wherein said clustering causes a net magnetic moment;
scanning said body for said ferromagnetic particles that have clustered around the target cells; and
identifying said clustered ferromagnetic particles based on said net magnetic moment.

9. A method as recited in claim 8, wherein said biological material comprises a monoclonal antibody.

10. A method as recited in claim 8, wherein said target cells comprise cancer cells.

11. A method for imaging cells in the body, comprising:
introducing cell targeting ferromagnetic nanoparticles into the body; and
scanning the body for a magnetic signature of a cluster of the ferromagnetic particles in relation to diamagnetic signature of the body; and
generating an image corresponding to said body scan.

12. A method as recited in claim 11, further comprising attaching a biological material to a plurality of said nanoparticles.

13. A method as recited in claim 12, wherein said biological material comprises a monoclonal antibody.

14. A method as recited in claim 12, wherein said biological material is attached to said nanoparticles with a bonding agent.

15. A method as recited in claim 12, wherein said biological material seeks out target cells in the body, such as cancer cells.

16. A method as recited in claim 15, wherein said ferromagnetic nanoparticles cluster around said cells.

17. A method as recited in claim 16, wherein said clustering causes a net magnetic moment.

18. A method for locating cells in the body, comprising:
attaching a biological material to ferromagnetic nanoparticles with a bonding agent;
introducing said cell targeting nanoparticles into the body;
wherein said biological material seeks out target cells in the body and causes said ferromagnetic nanoparticles cluster around said cells;
wherein said clustering causes a net magnetic moment;
scanning said body for said ferromagnetic particles that have clustered around the target cells; and
generating an image corresponding to said body scan.

19. A method as recited in claim 18, wherein said biological material comprises a monoclonal antibody.

20. A method as recited in claim 18, wherein said target cells comprise cancer cells.

21. An apparatus for locating cells in the body, comprising:
a magnetic body scanner configured for scanning the body for a magnetic signature of a cluster of cell targeting ferromagnetic nanoparticles in relation to a diamagnetic signature of the body.

22. A method as recited in claim 1, wherein a biological material is attached to a plurality of said nanoparticles.

23. A method as recited in claim 22, wherein said biological material comprises a monoclonal antibody.

24. A method as recited in claim 22, wherein said biological material is attached to said nanoparticles with a bonding agent.

25. A method as recited in claim 22, wherein said biological material seeks out target cells in the body, such as cancer cells.

26. A method as recited in claim 25, wherein said ferromagnetic nanoparticles cluster around said cells.

27. A method as recited in claim 26, wherein said clustering causes a net magnetic moment.

28. An apparatus for locating cells in the body, comprising:
a magnetic body scanner; and
a plurality of cell targeting ferromagnetic nanoparticles;
wherein said body scanner is configured for sensing the net magnetic moment generated by a cluster of said ferromagnetic nanoparticles introduced into the body;
wherein a biological material is attached to ferromagnetic nanoparticles with a bonding agent;
wherein said biological material seeks out target cells in the body and causes the ferromagnetic nanoparticles to cluster around those cells; and
wherein said clustering causes a net magnetic moment.

29. A method as recited in claim 28, wherein said biological material comprises a monoclonal antibody.

30. A method as recited in claim 28, wherein said target cells comprise cancer cells.

31. An apparatus for imaging cells in the body, comprising:
a magnetic body scanner configured for scanning the body for a magnetic signature of a cluster of cell targeting ferromagnetic nanoparticles in relation to a diamagnetic signature of the body; and
means for generating an image based on the magnetic signature of a cluster of nanoparticles.

32. An apparatus as recited in claim 31, a biological material is attached to a plurality of said nanoparticles.

33. An apparatus as recited in claim 32, wherein said biological material comprises a monoclonal antibody.

34. An apparatus as recited in claim 32, wherein said biological material is attached to said nanoparticles with a bonding agent.

35. An apparatus as recited in claim 32, wherein said biological material seeks out target cells in the body, such as cancer cells.

36. An apparatus as recited in claim 35, wherein said ferromagnetic nanoparticles cluster around said cells.

37. An apparatus as recited in claim 36, wherein said clustering causes a net magnetic moment.

38. An apparatus for imaging cells in the body, comprising:
a magnetic body scanner;
said magnetic body scanner configured for sensing the net magnetic moment generated by a cluster of ferromagnetic nanoparticles introduced into the body;
wherein a biological material is attached to ferromagnetic nanoparticles with a bonding agent;
wherein said biological material seeks out target cells in the body and causes the ferromagnetic nanoparticles to cluster around those cells; and
wherein said clustering causes a net magnetic moment; and
means for generating an image based on the net magnetic moment created by a cluster of nanoparticles.

39. An apparatus as recited in claim 38, wherein said biological material comprises a monoclonal antibody.

40. An apparatus as recited in claim 39, wherein said target cells comprise cancel cells.

41. A magnetic body scanner, comprising:
a magnetic flux measuring device; and
means for generating a line scan of the magnetic signature of the length of the body passing through the magnetic flux measuring device;
wherein the location of clusters of ferromagnetic particles introduced into the body can be identified from said line scan.

42. An apparatus as recited in claim 41, wherein a biological material is attached to a plurality of said ferromagnetic particles.

43. An apparatus as recited in claim 42, wherein said biological material comprises a monoclonal antibody.

44. An apparatus as recited in claim 42, wherein said biological material is attached to said nanoparticles with a bonding agent.

45. An apparatus as recited in claim 42, wherein said biological material seeks out target cells in the body.

46. An apparatus as recited in claim 45, wherein said target cells comprise cancer cells.

47. An apparatus as recited in claim 41, wherein said ferromagnetic nanoparticles cluster around said cells.

48. A method as recited in claim 47, wherein said clustering causes a net magnetic moment.

49. A magnetic body scanner, comprising:
a plurality of magnetic flux measuring devices;
a split coil, preferably of a superconducting type with the coil split into two windings, associated with each said magnetic flux measuring device, wherein the windings are counterwound in relation to each other; and
a conveyor or other device configured for moving a patient over the block and through the coils to perform a line scan;
wherein, as the patient gets scanned across the table, the incoming signal produces a broad image of the body; and
wherein, when ferromagnetic nanoparticles in the body are scanned, series of spikes are produced with amplitudes that are a function of the depth in the body.

50. A magnetic body scanner as recited in claim 49, wherein said magnetic flux measuring devices are selected from the group consisting essentially of SQUIDs, Flux Gate Magnetometers, and GMR magnetometers.

51. A magnetic body scanner as recited in claim 49, wherein a biological material is attached to a plurality of said ferromagnetic nanoparticles.

52. A magnetic body scanner as recited in claim 51, wherein said biological material comprises a monoclonal antibody.

53. A magnetic body scanner as recited in claim 51, wherein said biological material is attached to said nanoparticles with a bonding agent.

54. A magnetic body scanner as recited in claim 51, wherein said biological material seeks out target cells in the body.

55. A magnetic body scanner as recited in claim 54, wherein said target cells comprise cancer cells.

56. A magnetic body scanner as recited in claim 51, wherein said ferromagnetic nanoparticles cluster around said target cells.

57. A magnetic body scanner as recited in claim 56, wherein said clustering causes a net magnetic moment.
